(19) United States
(12) Patent Application Publication
(10) Pub. No.: US 2014/0295515 A1
(43) Pub. Date: Oct. 2, 2014

(54) CRUDE GLYCEROL FERMENTING PROCESS FOR THE PRODUCTION OF ETHANOL AND HYDROGEN

(71) Applicant: Cristiano Varrone, Roma (IT)

(72) Inventor: Cristiano Varrone, Roma (IT)

(21) Appl. No.: 14/344,601

(22) PCT Filed: Sep. 12, 2012
(86) PCT No.: PCT/IT2012/000281

§ 371 Date: Jun. 4, 2014

(30) Foreign Application Priority Data
Sep. 13, 2011 (IT) RM2011A000480

(51) Int. Cl. C12P 7/06; C12P 7/14

(52) U.S. Cl. CPC C12P 7/065; C12P 7/14
USPC 435/162; 435/161

(57) ABSTRACT

The present invention concerns a fermenting process of crude glycerol derived from biodiesel production for preparation of ethanol and hydrogen using activated sludge enriched and directly acclimatized on biodiesel by-product

The present invention concerns a crude glycerol fermenting process for production of ethanol and hydrogen. In particular, the invention concerns a crude glycerol fermenting process, said glycerol being derived from biodiesel production, for preparation of ethanol and hydrogen using activated, enriched and directly acclimatized on biodiesel by-product sludge.

The world-wide production of biodiesel in the past few years displayed an exponential type increase (European Biodiesel Board), resulting in a remarkable accumulation of crude glycerol as co-product. By now crude glycerol annually produced by biodiesel industry is approximately two fold than pure one that is consumed by pharmaceutical and cosmetic industries (Fonseca Amaral et al., 2009). Such production surplus resulted in price crash, such that today the chemical purification of crude glycerol costs up 6-7 times more than what is possible to be earned by sale. Therefore the chemical purification of crude glycerol is currently too much expensive, above all for small-medium companies (http://www.biodieselmagazine.com). Accordingly the development of new processes for low cost bio-conversion of glycerol in high added value products is considered a winning strategy in order to reduce the costs of biodiesel production (Dharmadi et al., 2006; Seifert et al., 2009) and obtain an energetic exploitation of glycerol.

Crude glycerol derived from biodiesel industry contains some contaminants (like methanol, soaps, salts, etc) such that direct use thereof by the industries is not allowed, as well as microorganism growth for bio-technological applications is also inhibited. The reduction of the pre-treatment would allow transformation costs remarkably to be reduced.

Various processes aiming at purification of crude glycerol are also known, in order pure product to be sold to pharmaceutical and cosmetic industry. However, currently, produced crude glycerol surplus results in a not cost-effective purification process.

In the light of above it is therefore apparent the need to provide for new processes suitable to overcome the disadvantages of prior art ones.

Recently therefore new processes for bio-conversion of glycerol in high added value products have been developed in order to reduce the production costs of biodiesel (Dharmadi et al., 2006; Seifert et al., 2009). The majority of the present intense literature activities focused on a biological process allowing crude glycerol to be degraded in 1,3-propanediol (Barbirato et al., 1998; Papanikolaou et al., 2000; Zheng et al., 2008). Not much work has been done about fermentation of glycerol in hydrogen (Liu & Fang, 2007 uses crude glycerol; Seifert et al., 2009 instead uses pure glycerol) or methane (Siles Lopez et al., 2009; Yang et al., 2008), and still less about potential conversion thereof in ethanol and hydrogen (Ito et al., 2005). Other studies moreover have displayed the possibility pure glycerol to be fermented in butanol (Biebl 2001), propionic acid (Bories et al., 2004) as well as ethanol and formic acid (Jarvis et al., 1997).

Processes for bio-conversion of glycerol in 1,3-propanediol (for example U.S. Pat. No. 6,890,451) and commercialization thereof are known, for example Shell and DuPont developed Corterra® named and Sorona® and Hytrel® named polymers, respectively.

In addition to 1,3-propanediol, the possibility glycerol to be fermented using a pure strain of Escherichia strains, following the metabolic pathway of 1,2-propanediol (KR 20080109787 (A)—Anaerobic fermentation of glycerol), has been displayed Another process concerns specific enzyme use, sequentially used in 4 separated reactors, in order crude glycerol to be degraded enzymatically (U.S. Pat. No. 7,601,524 (B1)—Commercial production of synthetic fuel from bio-diesel by products system). Again, in order crude glycerol to be treated, a process involving the fermentation by thermophilic microorganisms has been developed (JP 2008023426 (A)—Treatment method of crude glycerol), while some processes involve the use of various mixed substrates, among which there are also glycerol, in order butanol to be fermented (US 2009275787 (A1)—Alcohol production process) or mix of lipids, fat acids and hydrocarbons (EP 2313512 (A2)—Transformation of glycerol and cellulosic materials into high energy fuels).

Of course there are various processes for not biological processing of crude glycerol, like distillation (RU 2210560 (C1)—Method of distillation of crude glycerol) or hydrogen production by pyrolysis, after removal of contaminants (DE 102006056641 (A1)—Hydrogen production from glycerol, especially crude glycerol from biodiesel production, involves removing contaminants, pyrolyzing and separating hydrogen from carbon monoxide in product).

The authors of the present invention now have developed a new process of crude glycerol fermentation for hydrogen and ethanol preparation, using enriched sludge as inoculum. For the first time crude glycerol derived from production of biodiesel has been used, in order to optimize at the same time both the production and yield of bioethanol and biohydrogen, using as fermentation inoculum enriched and directly acclimatized on waste biodiesel activated sludge. Moreover for the first time it has been possible an efficient substrate degradation without the use of complex culture media, i.e. containing vitamins, mineral elements, tryptone and yeast extract, to be obtained.

The process of the present invention allows crude glycerol to be transformed by microbial fermentation in other energetic vectors, like ethanol and hydrogen, without expensive pre-treatments of substrate. Ethanol production is estimable in over than 500 kg/t of glycerol, and the same can be directly used (or by blending with biodiesel), while hydrogen, produced in amount of 260 m³/t of glycerol, can be used as high value energetic vector, supplying energy required for process operation.

The process has been developed by selection of a specific microbial pool (activated enriched sludge) and statistical optimization of process parameters.

They are not many bacteria suitable to carry out glycerol fermentation and, moreover, the process efficiency is subjected to remarkable drop when crude (by-product from biodiesel industry) is substituted for pure glycerol. Crude glycerol contains in fact various contaminants, like methanol, as residues of soap and salts deriving from trans-esterification process, inhibiting the bacterial growth. The use of “functional consortium” according to the present invention, selected and acclimatized directly on such substrate, allowed on the contrary a pool of together operating bacteria, guaranteeing neutralization of contaminants and fermentation results similar to best ones of pure glycerol, to be obtained. Moreover, the use of statistical optimization is generally successfully applied to pure strains and not to mixed pools. However, the use of stable functional consortium (that from functional the point of view behaves as a pure strain) according to the present invention allowed highly meaningful statistical models to be obtained.

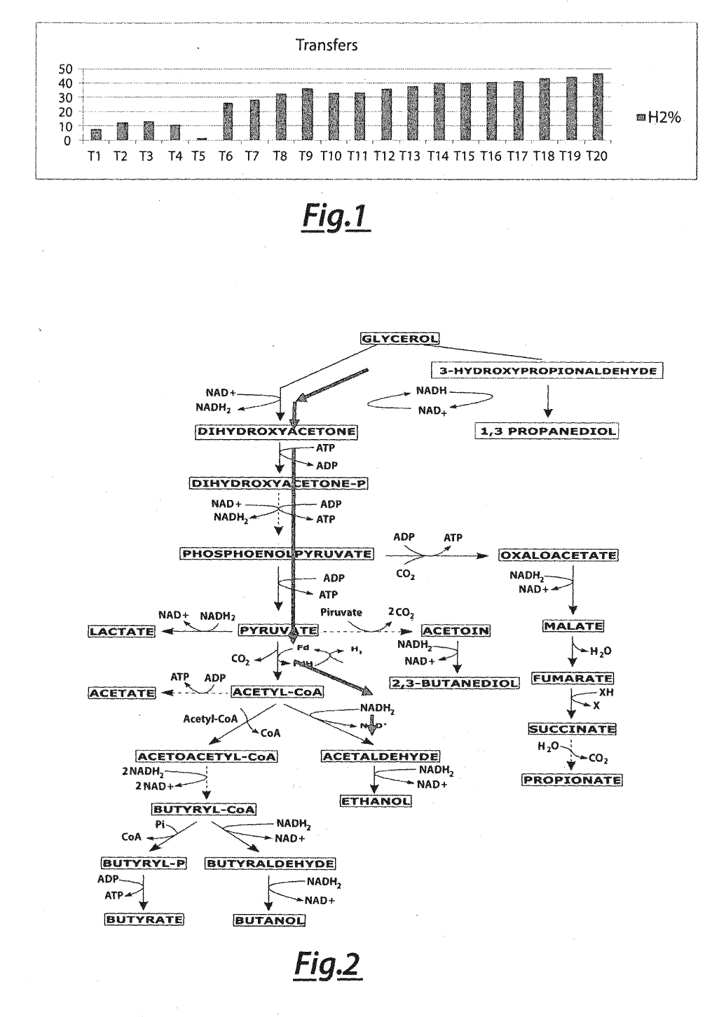

Another technical problem resolved by the present invention is the inhibition observed at the beginning of enrichment, after 4-5 transfers (see FIG. 1), when the consortium is still not substrate acclimatized. Such phenomenon could be derived from metabolite accumulation inhibiting bacterial growth and which metabolites are transferred and accumulated at every passage. The problem is resolved using “pellet material”, instead of fermentation liquid as such. Thus bacteria again recovered their growth since first passage (FIG. 1; T6) and it has been possible the enrichment to be completed, without no more inhibition phenomena appearance.

Finally an important problem resolved by the present invention is the possibility to optimize both yield and production (of fermentation) at the same time. In literature currently referred studies generally the optimization of either yield or production is reported, both values not being reported together. This because often the conditions generally enhancing an efficient degradation of substrate and good yield (typically low substrate concentrations) result in low production, while elevated substrate concentrations result in production increase, but lower degradation efficiency of substrate (and therefore low yields) (Wang & Wan, 2008). Due to the use of experimental design of the invention, on the other hand, it has been possible the whole process to be optimized, by detecting conditions according to which at the same time both the yield and production of ethanol and hydrogen are maximized. However high values of determination coefficient (R²>0.96, explaining therefore 96% of the variability of experimental data) and yields near to theoretical ones both for hydrogen and ethanol, concurrently with maximum production, clearly indicate the successful operation of the model and effectiveness of such an approach.

As above reported, up to now there are least studies on the hydrogen production from fermentation of crude glycerol, and still less as to ethanol. The bio-conversion of crude glycerol is generally carried out to produce 1,3-propanediol, that is a compound at high added value used by textile industries for synthesis of biodegradable polymers in textile fibre production. This process allows large substrate amounts (generally more than 50 g/L) to be converted, however the Italian biodiesel industry displayed an insufficient interest about the possibility to convert crude glycerol in high added value products different than biofuel, preferring, currently, the waste combustion. On the contrary interest was displayed about possibility waste therefrom to be converted in bio-ethanol, to be sold on the market, to be added directly to biodiesel (allowing performances to be improved), or as methanol substitute in biodiesel trans-esterification.

Of course one of the main advantages of the process according to the invention is to meet such a requirement, being selected a metabolic pathway producing hydrogen by an “ethanol type fermentation”, aiming, among other things, at cost lowering, thanks to a targeted trick set and better waste degradation.

Double combined hydrogen and bioethanol optimization allows in fact to use hydrogen for at least heating fermentation device, in addition to obtain the ethanol to be added to biodiesel. According to an up-scaling operation, the hydrogen production is estimable around 260 m³ in 44 hours, for every ton of glycerol, supplying 921 kWh in 44 hours, i.e. 21 kW (considering a heat of combustion of 286 kJ/mol). In fact, a 50 m³, fermentation device provided with 2 cm thick commercial thermal insulation (0.04 W/m·K exchange coefficient) would have a dispersion variable from 0.8 to 3.3 KW, with external temperatures from −5 to 25° C. For starting up, instead, about 700 kWh would be necessary in order to heat 50 m³ of water from 25 to 37° C. It is therefore apparent that this process allows a major part of energy used for the fermentation to be recovered.

The selection of functional consortium according to the invention (activated sludge enriched and acclimatized mixed pool) has allowed efficiently crude glycerol to be degraded (see hydrogen and ethanol yields), avoiding expensive pre-treatments and use of complex media containing solutions of vitamins and trace elements, tryptone or yeast extract. The use of such media should be avoided in order costs and fermented product disposal problems to be reduced, but in fact all the research studies continue the use thereof in order to favour the growth of bacterial biomass and enhance the substrate degradation. In addition, again in order the costs to be reduced and operations to be simplified, optional anaerobic bacteria have selected, thus avoiding operation under full anoxia conditions. The result has been a very strong and easy to use microbial pool, surviving even if the bacteria are transferred into the air or left for a long time at room temp in the fermented media.

The advantage of this process is that it allows best substrate conversion yield to be obtained, i.e. obtaining 2.5 times more ethanol than up to now reported under same operating conditions in literature (and consuming 50% more substrate), lowering operating costs. Moreover, higher substrate degradation allows smaller size fermentation to be used in up-scaling operation, thus further reducing plant costs.

It is to be pointed in addition that the bio-ethanol production from crude glycerol results in remarkable advantages compared to normally employed substrates (various origin carbohydrates). In fact, glycerol displays higher reduction degree thus allowing higher yields of reduced products to be obtained (Dharmadi et al., 2006). Moreover, ethanol producing plants from, for example, ligno-cellulosic or starch rich substrates (maize), are very more complex, involving operating costs (and also as plant investment costs) higher up to 35-40% (Yazdani and Gonzalez, 2007).

Ito and co-workers reports a 0.71 mol H2/mol glycerol yield and <067 mol EtOH/mol glycerol for substrates at concentrations >10 g/L of crude glycerol (using 5 g/L of tryptone and 5 g/L of yeast extract). Using the process according to the present invention it has been possible to degrade completely 20 g/L of crude glycerol and under optimized conditions, without addition of vitamins, mineral elements, tryptone and yeast extract. It has been possible in addition to degrade over 15 g/L of crude glycerol with H2 and ethanol yields near to 1 mol/mol glycerol (i.e. theoretical yield).

It is therefore a specific object of the present invention a crude glycerol fermenting process for the production of ethanol and hydrogen, said process comprising or consisting of the following steps:

A) diluting said crude glycerol with culture media for bacterial fermentation;

B) inoculating said crude glycerol of step A) with inoculum containing bacteria suitable for glycerol fermentation, selected from material containing said bacteria and acclimatized directly on crude glycerol;

C) fermenting under anaerobic conditions until no further increase of hydrogen percentage and/or substrate consumption is observed.

In order to lower the production costs, it is possible also to use poor culture media such that crude glycerol is unique carbon source.

Particularly, said inoculum containing bacteria suitable for glycerol fermentation selected from material containing said bacteria and acclimatized directly on crude glycerol is obtained using the following steps of:
a) preparing culture media wherein the unique carbon source is crude glycerol and said crude glycerol is diluted until to obtain a concentration of contaminants contained in crude glycerol such that the growth of said bacteria suitable for glycerol fermentation is not inhibited;
b) inoculating one or more sample of material containing bacteria suitable for glycerol fermentation in a correspondent sample number of said culture media;
c) fermenting under anaerobic conditions;
d) after a fermentation period such that further increase of hydrogen percentage and/or substrate consumption is not detected, withdrawing a fermented aliquot from the sample producing higher percentage of biogas and/or hydrogen, transferring said aliquot in fresh culture media and repeating the fermentation;
e) withdrawing a fermented aliquot from step d) in the late phase of maximum bacteria growth rate and transferring said aliquot in fresh culture media;
f) repeating step e) until further volumetric hydrogen production increase is not detected

The process of bacteria mixture selection is carried out using material that contains bacteria suitable for glycerol fermentation. Suitable materials can be of various type, for example activated sludge, as for example activated sludge from sewage water purifier, sludge from oil industry or rich lipid sludge, sewage from beer or dairy fermentation processes, vegetal sewage, lagoon sediments. Those skilled in the art, starting from a material possibly containing bacteria suitable to degrade glycerol, are able to verify the effective presence of desired bacteria simply by means of preliminary tests in culture media containing, like unique carbon source, glycerol. After material selection, the selection process of bacteria mixture according to the invention is suitable to enhance the ability thereof for glycerol degradation and both hydrogen and ethanol production.

Most suitable culture media for the selection of microorganisms can be selected by means of simple preliminary tests. As mentioned above, the selection of the mixture of bacteria is carried out using crude glycerol as unique carbon source, said crude glycerol being diluted with culture media containing as little as possible nutrient for growth of glycerol degrading bacteria. For example, a suitable culture media can contain essential elements like magnesium, nitrogen, phosphorus and sulfur and trace elements like magnesium, iron, calcium necessary for cell physiology and possibly vitamins and/or tryptone. Culture media will be therefore a selective media enhancing the growth only of microorganisms having desired function, that is glycerol degradation and combined hydrogen and ethanol production. A specific example of culture media is described in example 1.

The process of bacteria selection can be carried out using one or more sample. By using more than one material sample it is possible to enhance the probability to detect sample containing bacteria with greater ability to degrade glycerol in as little as possible time.

Crude glycerol can contain various glycerol concentrations depending on the process used for biodiesel production. Concentration can be from 55 to 90%, preferably from 80 to 90%. Based on type of crude glycerol used both percentages and type of contaminants will be different, as for example citric or sulphuric acid, methanol, sodium acetate, soaps, etc. Contaminants can inhibit the growth of bacteria and thus also those having desired function according to the present invention. Therefore, the dilution of crude glycerol will be established based on type of used glycerol in order to dilute contaminants and inhibit negative effect thereof on bacteria growth, considering the final concentration of glycerol, that must be a sufficient carbon source. Most suitable dilution based on type of crude glycerol used can be identified using dilution preliminary tests.

As above described, the selection of the mixture of bacteria displaying stable function in degrading glycerol is carried out repeating many times step e), that is withdrawing a fermented aliquot of step d) in the late phase of maximum bacteria growth rate and transferring said aliquot in fresh culture media.

The identification of the late phase of maximum bacteria growth rate consists of a process well known by those skilled in the art. Accordingly a kinetic study is carried out wherein the fermentation is controlled until the end of the process is reached, that is when biogas production and substrate consumption no more occur, recording hydrogen production data at regular intervals. Then said data will be used to make a logistic curve supplying information about bacterial growth and characterizing the process kinetics. Once logistic curve has been plotted, the point of maximum growth (inflexion point) and time period necessary to reach the same will be detected. This time period will be the reference minimal value. In fact the transfer of the aliquot must be carried out in a step immediately successive to maximum growth, before to reach the curve asymptote (the plateau).

According an alternative embodiment, in order to enhance the growth of bacteria displaying desired function, as an alternative to step e) can be carried out step e′) by withdrawing a fermented aliquot from step d) in the late phase of maximum bacteria growth rate, centrifuging said aliquot in order bacteria to be settled and transferring said settled material (pellet) containing the bacteria in fresh media, supernatant being discarded.

The mixture of bacteria displaying stable glycerol degradation activity can be stored at −18° C. for several months. Before the use in the production of ethanol and hydrogen, it will be thawed and pre-activated by fermentation over few hours in order exponential growth phase to be reached.

The culture media of phase A) is a culture media for bacterial fermentation, not necessarily it will be a minimal culture media as used for bacteria selection.

The crude glycerol according to the present invention is preferably a by-product of biodiesel production.

The fermentation, both for ethanol and hydrogen production and bacteria selection processes according to the present invention, can be carried out at temperatures from 10 to 41° C., preferably 35 to 40° C., more preferably from 37 to 38° C., while pH can be from 5 to 10, preferably from 6 to 9, more preferably from 7.8 to 8.2.

Where crude glycerol contains approximately from 80% to 90% of glycerol, said crude glycerol, both for ethanol and hydrogen production and bacteria selection processes according to the present invention, can be diluted in such way to obtain from 0.5 to 100 grams of glycerol for litre of final reaction mixture (working solution), preferably from 1 to 20 g/L, more preferably from 15 to 16 g/L. Moreover, said inoculum containing bacteria suitable for glycerol fermentation can be from 0,5 to 50% by volume of the final reaction mixture total volume (total working volume), preferably from 0.5 to 20%, more preferably from 5 to 10%.

The present invention further is directed to a process for selection of bacteria suitable for fermentation of glycerol from material containing said bacteria and acclimatization thereof directly on crude glycerol as above defined.

The present invention now will be described by an illustrative, but not limitative way, according to preferred embodiments thereof with particular reference to enclosed drawings, wherein:

FIG. 1 shows the growth of biogas contained H₂ percentage during transfer (T) of best batches during the enrichment of activated sludge.

FIG. 2 shows in boldface the main metabolic pathway.

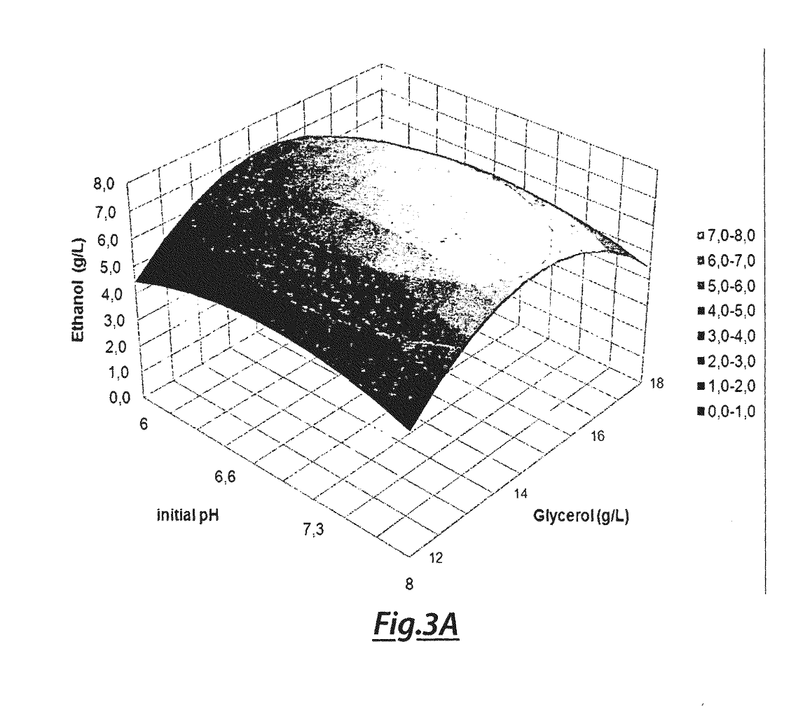

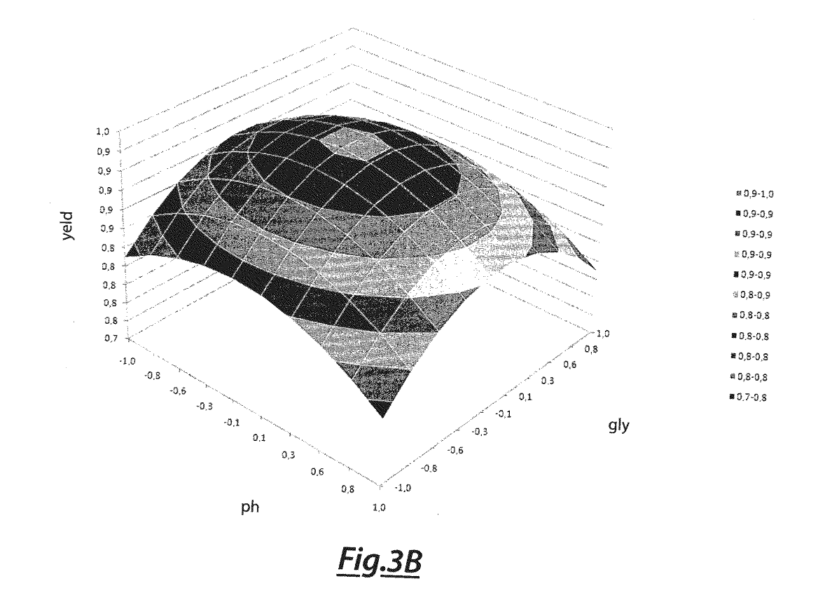

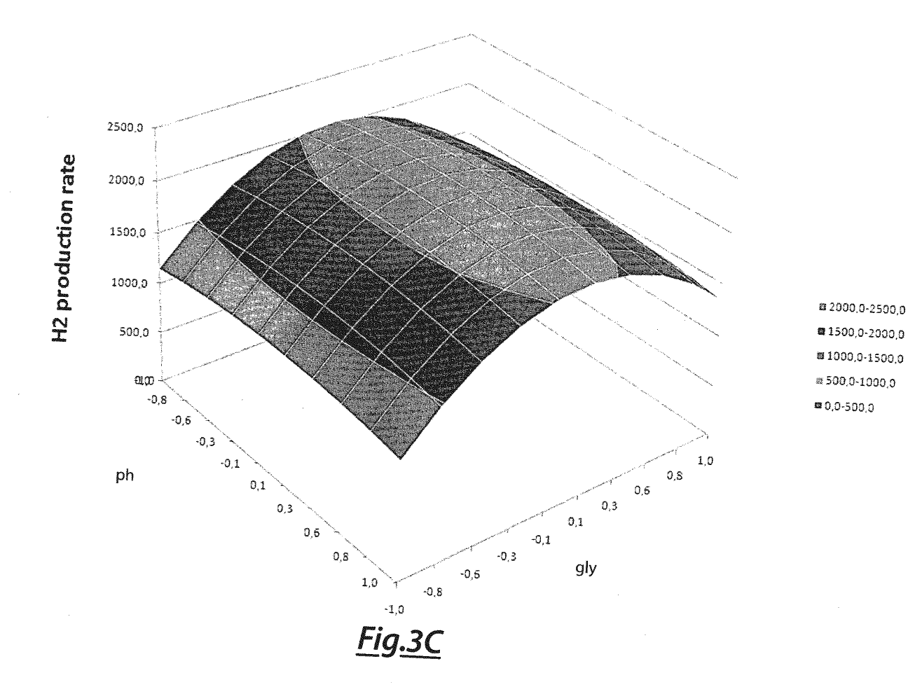

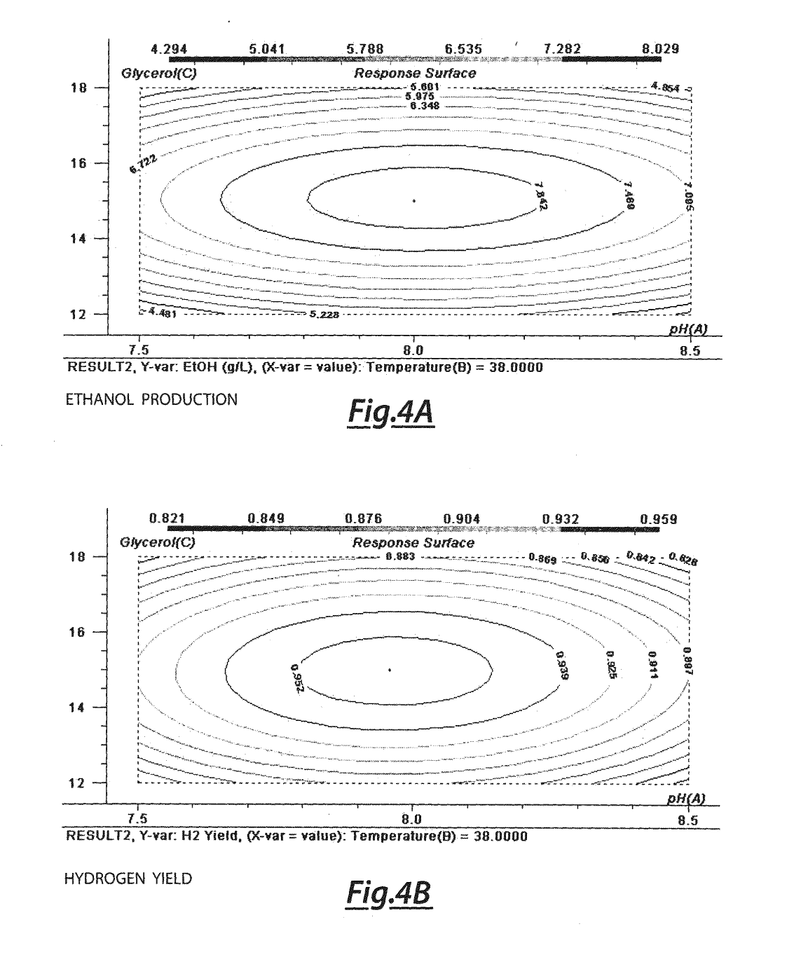

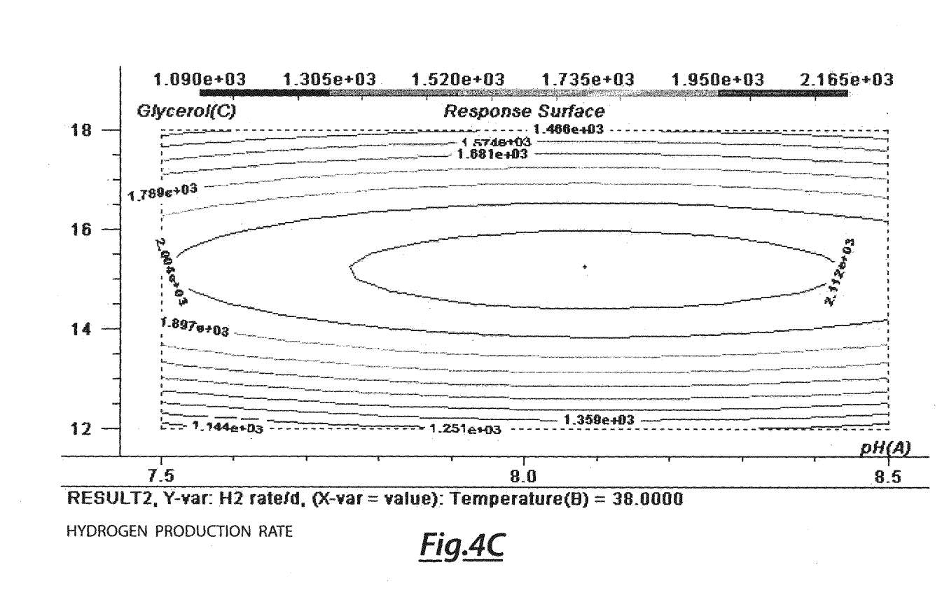

FIGS. 3 and 4 show respectively three-dimensional and bidimensional diagrams of response surfaces obtained using Box-Behnken Design for the optimization of ethanol production in g/L. A: Hydrogen Yield (mol H2/mol consumed glycerol) with pH variation and glycerol concentration, keeping temperature at an optimal value. B: Hydrogen production rate (ml H2/L fermentation device/day) with pH variation and glycerol concentration, keeping temperature at an optimal value. C: Ethanol production (g/L) with pH variation and glycerol concentration, keeping temperature at an optimal value.

### EXAMPLE 1

### Development of the Process of Active Sludge Enrichment According to the Invention and Optimization of Process for Ethanol and Hydrogen Preparation from Biodiesel Crude Glycerol

Before the statistical optimization of process for ethanol and hydrogen preparation from crude glycerol, an at hoc microbial pool (defined “functional consortium”, sensu (Adav et al., 2009)) is selected using a process applied for the first time by the authors of the present invention to biodiesel crude glycerol and appropriately modified. Said process allowed an efficient conversion of crude glycerol in ethanol and hydrogen. Such microbial pool has been obtained using microbiological techniques of activated sludge enrichment and acclimatization, under very selective conditions, resulting in a process of stable fermentation (conditio sine qua non in order statistical optimization of process using a mixed pool to be used). Said sludge has been withdrawn from sewage purifier of Harbin city, in the Heilongjiang province in China.

Fermentation conditions used were as below:

| Batch Volume | 150 | mL |
|---|---|---|
| Culture media volume | 45 | mL |
| Inoculum | 5 | mL |
| Initial pH | 6.8 | |
| Temperature | 37° | C. |
| stirring | 150 | rpm |

A litre of culture media contains:
20 g glycerol
3.4 g K₂HPO₄×3H₂O
1.3 g KH₂PO₄
2 g (NH4)₂SO₄
0.2 g MgSO₄×7H₂O
20 mg CaCl₂×2H₂O
5 mg FeSO₄×7H₂O

Through a series of fast passages in fresh media, activated sludge enrichment has obtained, using very poor culture media, crude glycerol being unique carbon source. Before the incubation in thermostat bath, the batches were flushed for 3 minutes with argon, in order anaerobic conditions to be generated. It has been chosen not to use nitrogen (commonly used and less expensive) in order to avoid at higher extent selection of nitrogen-fixer bacteria. At 21 hour intervals 10% (v/v) by volume of fermented material was withdrawn from the batch displaying best results for H₂ production and transferred in fresh media (3 times). In order to avoid metabolite inhibition problems, “pellet”, instead of fermentation liquid as such, has been used. Practically at every transfer 10% by volume of fermented material was withdrawn, then centrifuged at 10000 rpm for 5 minutes, such that the bacteria were settled on the bottom of a conical glass tube (15 mL) thus forming said pellet. At this time the supernatant (metabolite containing) was discarded and the pellet re-suspended in equivalent fresh media and inoculated in a new batch. All the operations are carried out under sterile conditions. Repeated passages resulted in an enrichment of species succeeding to grow quickly on such substrate, selecting moreover the best hydrogen producers. Thus it has been possible to increase H₂ percentage from the 7 to 46% (FIG. 1). This has been possible because the fast passages (21 hours) allows from time to time to transfer only the bacteria growing more fast on crude glycerol. In such a way, at every transfer poor performance microorganisms are eliminated, resulting in an increasingly enrichment of best ones.

During this phase bacteria and conditions enhancing the metabolic ethanol pathway have been selected (“ethanol-type fermentation”; Renseneb et al., 1997) to the detriment of yield lowering 1,3-propanediol (FIG. 2). This has been possible thanks to the fact that the use of classic culture media containing nutrients and vitamins, thus favouring the production of 1,3-propanediol (synthesized by a vitamin B₁₂ dependent coenzyme), has been purposely avoided. All this allowed very high yields of hydrogen and ethanol production to be obtained.

Under continuous transfer, the conditions remained stable, resulting in a total biogas production between the 230 and 280 mL, and H₂ percentage higher than 40% (approximately production of 100 mL of H₂ from 50 mL of media).

Then the microbial pool has been frozen in 15 ml Falcon tube at −18° C. in order to be stored.

Successively, before of each experiment, 3 samples have been thawed by keeping the same for 4 hours in refrigerator (at 4° C.) and then 1 hour at room temp., before to be inoculated. Afterwards said samples have been incubated for 24 hours (pre-activation) and only the best batch transferred in fresh media. The minimal conditions for the transfer are considered the production of 220 ml of biogas with at least 35% of hydrogen in 24 hours.

Statistical Optimization of the Process for Ethanol and Hydrogen Production:

After the selection of “functional consortium” (under stable and consistent conditions of fermentation) it has been possible to carry out a statistical optimization of the process parameters, so as to maximize both the yield and the ethanol and hydrogen production. At this end specific statistical designs have been used (Plackett-Burmann screening design and Box-Behnken Optimization Design), previously successfully applied in various studies of several substrate bio-conversion (Annadurai et al., 1999; Francis et al., 2003; Mu et al., 2006; Pan et al., 2008; Long et al., 2010).

Plackett-Burmann design allowed the effect of main fermentation parameters on hydrogen bioproduction to be estimated. Table 1 shows variables and corresponding ranges estimated using Plackett-Burmann design.

TABLE 1

| | | Low | High | | | |
|---|---|---|---|---|---|---|
| | | level | level | | | |
| Code | Variable | (−1) | (+1) | F-Ratio | B coeff | p value |
| X₁ | Glycerol (g/L) | 5 | 15 | 7.727 | −8.633e−03 | 0.0195* |
| X₂ | Initia pH l | 6 | 8 | 6.452 | 3.936e−02 | 0.0296* |
| X₃ | Temperature | 35 | 39 | 28.105 | 4.116e−02 | 0.0003* |
| | (° C.) | | | | | |
| X₄ | Yeast extract | 0.5 | 1.5 | 2.881 | −5.271e−02 | 0.1205 |
| | (g/L) | | | | | |
| X₅ | Tryptone (g/L) | 0.5 | 1.5 | 0.478 | 2.146e−02 | 0.5052 |

*p < 0.05.

According to results the variable with statistically meaningful effect on the process (p<0.05) are glycerol, initial pH and temperature. It has been therefore possible to demonstrate on statistical base that yeast extract and tryptone were not necessary in our process. This has been possible thanks to the pool of bacteria selected and acclimatized on very selective synthetic substrate.

In the successive step Box Behnken design allowed ranges of meaningful parameters, previously characterized using Plackett-Burmann design, to be optimized. The experimental results are reported in table 2 that shows the effect of glycerol, temperature and initial pH on the rate production of H2, yield thereof and ethanol production.

TABLE 2

| | Glycerol | | | | Temp | | H₂ | H₂ | |
|---|---|---|---|---|---|---|---|---|---|
| | (g/L) | | pH | | (° C.) | | Yield | Rate | Ethanol |
| Run | X₁ | X₁ code | X₂ | X₂ code | X₃ | X₃ code | molH₂/mol gly | H₂ mL/L/d | g/L |
| 1 | 15 | 0 | 7.5 | − | 37 | − | 0.93 | 2013.2 | 6.63 |
| 2 | 15 | 0 | 8.5 | + | 37 | − | 0.91 | 2203.0 | 6.95 |
| 3 | 15 | 0 | 7.5 | − | 39 | + | 0.88 | 1778.2 | 7.28 |
| 4 | 15 | 0 | 8.5 | + | 39 | + | 0.86 | 1840.9 | 6.90 |
| 5 | 12 | − | 7.5 | − | 38 | 0 | 0.82 | 1147.5 | 4.12 |
| 6 | 12 | − | 8.5 | + | 38 | 0 | 0.79 | 1205.8 | 4.37 |
| 7 | 18 | + | 7.5 | − | 38 | 0 | 0.84 | 1298.2 | 4.42 |
| 8 | 18 | + | 8.5 | + | 38 | 0 | 0.82 | 1321.0 | 4.85 |
| 9 | 12 | − | 8 | 0 | 37 | − | 0.90 | 1226.9 | 5.55 |
| 10 | 12 | − | 8 | 0 | 39 | + | 0.86 | 1090.0 | 5.15 |
| 11 | 18 | + | 8 | 0 | 37 | − | 0.89 | 1453.1 | 5.44 |
| 12 | 18 | + | 8 | 0 | 39 | + | 0.83 | 1379.6 | 5.40 |
| Centre 1 | 15 | 0 | 8 | 0 | 38 | 0 | 0.94 | 2158.4 | 7.98 |
| Centre 2 | 15 | 0 | 8 | 0 | 38 | 0 | 0.95 | 2166.6 | 7.86 |
| Centre 3 | 15 | 0 | 8 | 0 | 38 | 0 | 0.95 | 2170.1 | 7.97 |
| Centre 4 | 15 | 0 | 8 | 0 | 38 | 0 | 0.94 | 2122.7 | 7.92 |

Under optimized conditions therefore it has been possible to obtain in 44 hours approximately 8 g/L of ethanol (yield 1 mol EtOH/mol glycerol) from 15 g/L of glycerol and almost 2.2 L/L/d of hydrogen (0.95 mol H₂/mol glycerol, value near to theoretical yield, i.e. 1; Markov et al., 2011), with H₂ amount in biogas being about 50%.

Plackett-Burmann design therefore allowed parameters having a statistically meaningful effect on the process to be defined. Box-Behnken design further allowed values of meaningful parameters to be optimized, supplying moreover a mathematical model predicting the fermentation process, allowing the process to be studied and to estimate as production of ethanol and hydrogen will vary depending on variation of experimental parameters (FIGS. 3 and 4), with values of coefficient of determination (R²) higher than 0.96.

Being “ethanol-type fermentation” selected, obviously ethanol represents the main fermentation product (2/3), while the other metabolites as lactate, 1,3-propanediol, succinate and formate contribute in lower extent to final total fermented material (about 12 g/L).

Compared to initial conditions it has been possible to produce about 200 mL of H₂ from 50 ml of media, thus doubling the production.

The use of statistical optimization of a biological process on enriched sludge allowed both the yield and production of ethanol and hydrogen to be maximized, without chemical pre-treatment of the substrate in order to eliminate contaminants.

### BIBLIOGRAPHY

Adav S. S., Lee D. J., Wang A. J., Ren N. Q. 2009. Functional consortium for hydrogen production from cellobiose: Concentration-to-extinction approach. Bioresource Technology 100: 2546-2550.
Annadurai G., Balan S. M., Murugesan T. 1999. Box-Behnken design in the development of optimized complex medium for phenol degradation using Pseudomonas putida (NICM 2174). Bioprocess Eng. 21:415-421.
Barbirato, F., Himmi, E. H., Conte, T., Bories, A. 1998. 1,3-Propanediol production by fermentation: an interesting way to valorise glycerine from the ester and ethanol industries. Ind. Crops Prod. 7, 281-289.
Biebl, H. 2001. Fermentation of glycerol by Clostridium pasteurianum—batch and continuous culture studies. J. Ind. Microbiol. Biotechnol. 27, 18-26.
Bories, A., Himmi, E., Jauregui, J. J. A., Pelayo-Ortiz, C., Gonzales, V. A. 2004. Glycerol fermentation with Propionibacteria and optimisation of the production of propionic acid. Sci. Aliments. 24, 121-135.
Dharmadi Y., Murarka A, Gonzalez R. 2006. Anaerobic Fermentation of Glycerol by Escherichia coli: A New Platform for Metabolic Engineering. Biotechnology and Bioengineering, Vol. 94, No. 5. 821-829
European Biodiesel Board Statistics.
Fonseca Amaral P. F., Felix Ferreira T., Cardoso Fontes G., Zarur Coelho M. A. 2009. Glycerol valorization: New biotechnological routes. Food and Bioproducts Processing 87:179-186.
Francis F., Sabu A., Nampoothiri K. M., Ramachandran S., Ghosh S., Szakacs, G., Pandey A. 2003. Use of response surface methodology for optimizing process parameters for the production of a-amylase by Aspergillus oryzae. Biochem. Eng. J. 15:107-115.
Ito T., Nakashimada Y., Senba K., Matsui T., Nishio N. 2005. Hydrogen and ethanol production from glycerol-containing wastes discharged after biodiesel manufacturing process. Journal of bioscience and bioengineering. Vol 100, No 3; 260-265.
Jarvis, G. N., Moore, E. R. B., Thiele, J. H. 1997. Formate and ethanol are the major products of glycerol fermentation produced by a Klebsiella planticola strain isolated from red deer. J. Appl. Microbiol. 83, 166-174.
Liu F., and Fang B. 2007. Optimization of bio-hydrogen production from biodiesel wastes by Klebsiella pneumonia. Biotechnol. J., 2, 374-380.
Long C., Cui J., Liu Z., Liu Y., Long M., Hua Z. 2010. Statistical optimization of fermentative hydrogen production from xylose by newly isolated Enterobacter sp. CN1. Int J Hydrogen Energy 35:6657-6664.
Markov S. A., Averitt J., Waldron B. 2011. Bioreactor for glycerol conversion into H2 by bacterium Enterobacter erogene. International Journal of Hydrogen Energy 36: 262-266.
Mu Y., Wang G., Yu H. Q. 2006. Response surface methodological analysis on biohydrogen production by enriched anaerobic cultures. Enzyme and Microbial Technology 38:905-913.
Pan C. M., Fan Y. T., Xing Y., Hou H. W., Zhang M. L. 2008. Statistical optimization of process parameters on bio-hydrogen production from glucose by Clostridium sp. Fanp2. Bioresource Technology 99:3146-3154.
Papanikolaou S., Ruiz-Sanchez P., Pariset B., Blanchard F., Fick M. 2000. High production of 1,3-propanediol from industrial glycerol by a newly isolated Clostridium butyricum strain. Journal of Biotechnology 77, 191-208.
Ren N Q., Wang B., Huang J C. 1997. Ethanol-Type fermentation from carbohydrate in high rate acidogenic reactor. Biotechnol Bioeng, 54, 428-433.
Seifert K., Waligorska M., Wojtowski M., Laniecki M. 2009. Hydrogen generation from glycerol in batch fermentation Process. Int. J. Hydrogen Energy 34, 3671-3678.
Siles López J. A., Martin Santos M. A., Chica Perez A. F., Martin Martin A. 2009. Anaerobic digestion of glycerol derived from biodiesel manufacturing. Bioresour. Technol. 100 (23), 5609-5615.
Wang J. and Wan W. 2008. Optimization of fermentative hydrogen production process by response surface methodology. Int J Hydrogen Energy 33:6976-6984.
Yazdani S. S. and Gonzalez R. 2007. Anaerobic fermentation of glycerol: a path to economic viability for the biofuels industry. Current Opinion in Biotechnology 2007, 18:213-219.
Yingnan Yang Y., Tsukahara K., Sawayama S. 2008. Biodegradation and methane production from glycerol containing synthetic wastes with fixed-bed bioreactor under mesophilic and thermophilic anaerobic conditions. Process Biochemistry 43:362-367
Zheng Z-M., Hu Q-L., Hao J., Xu F., Guo N-N., Sun Y., Liu D-H. 2008. Statistical optimization of culture conditions for 1,3-propanediol by Klebsiella pneumoniae AC 15 via central composite design. Bioresource Technology 99; 1052-1056.

1. A crude glycerol fermenting process for the production of ethanol and hydrogen, said process comprising:
A) diluting said crude glycerol with culture media for bacterial fermentation;
B) inoculating said crude glycerol of step A) with an inoculum containing bacteria suitable for glycerol fermentation, the inoculum selected from a material containing said bacteria and acclimatized directly on crude glycerol as a unique carbon source;
C) fermenting under anaerobic conditions until no further increase of hydrogen percentage and/or substrate consumption is observed.

2. The process according to claim 1, wherein said inoculum containing bacteria suitable for glycerol fermentation selected from a material containing said bacteria and acclimatized directly on crude glycerol is obtained by:
a) preparing culture media comprising crude glycerol as a unique carbon source in which said crude glycerol is diluted to obtain a concentration of contaminants contained in the crude glycerol such that the growth of said bacteria suitable for glycerol fermentation is not inhibited;
b) inoculating one or more sample of the material containing bacteria suitable for glycerol fermentation in a correspondent sample number of said culture media;
c) fermenting under anaerobic conditions;
d) after a fermentation period such that further increase of hydrogen percentage and/or substrate consumption is not detected, withdrawing a fermented aliquot from a sample producing a higher hydrogen volume transferring said fermented aliquot in fresh culture media and repeating the fermentation;
e) withdrawing a fermented aliquot from step d) in a late phase of maximum bacteria growth rate and transferring said fermented aliquot from step d) in fresh culture media;
f) repeating step e) until further increase of biogas and/or hydrogen percentage is not detected.

3. The process according to claim 2, wherein alternatively to step e), step, e′) of
withdrawing a fermented aliquot from step d) in a late phase of maximum bacteria growth rate, centrifuging and transferring centrifugation settled material in a fresh culture media
is carried out.

4. The process according to claim 1, wherein the crude glycerol is a by-product of biodiesel production.

5. The process according to claim 1, wherein the material containing bacteria suitable for glycerol fermentation is selected from the group consisting of activated sludge, sewage from beer or dairy fermentation processes, vegetal sewage, and lagoon sediments or at high trophic condition.

6. The process according to claim 1, wherein the fermenting of crude glycerol is carried out at temperatures from 10° C. to 41° C.

7. The process according to claim 1, wherein the fermenting of crude glycerol is carried out at pH from 5 to 10.

8. The process according to claim 1, wherein, when crude glycerol contains approximately from 80% to 90% of glycerol, said crude glycerol is diluted in such way to obtain from 0.5 to 100 grams of glycerol for liter of mixture of final reaction.

9. The process according to claim 1, wherein, when the crude glycerol contains approximately from 80% to 90% of glycerol, inoculum containing bacteria suitable for glycerol fermentation varies from 0.5 to 50% by volume of final reaction mixture total volume.

10. A process of selection of bacteria suitable for glycerol fermentation from material containing said bacteria and acclimatization thereof on crude glycerol according to claim 2.

11. The process according to claim 1, wherein the fermenting of crude glycerol is carried out at temperatures from 35° C. to 40° C.

12. The process according to claim 1, wherein the fermenting of crude glycerol is carried out at temperatures from 37° C. to 38° C.

13. The process according to claim 1, wherein the fermenting of crude glycerol is carried out at pH from 6 to 9.

14. The process according to claim 1, wherein the fermenting of crude glycerol is carried out at pH from 7.8 to 8.2.

15. The process according to claim 1, wherein, when crude glycerol contains approximately 80% to 90% of glycerol, said crude glycerol is diluted in such way to obtain from 1 to 20 grams of glycerol for liter of mixture of final reaction.
